(19) United States
(12) Patent Application Publication
(10) Pub. No.: US 2009/0074672 A1
(43) Pub. Date: Mar. 19, 2009

(54) Tumor Boundary Imaging

(75) Inventors: Gregory W. Faris, Menlo Park, CA (US); Chia-Pin Pan, Menlo Park, CA (US); Zishan Haroon, Chadds Ford, PA (US)

Correspondence Address: RICHARD ARON OSMAN, 4070 CALLE ISABELLA, SAN CLEMENTE, CA, 92672, US

(73) Assignee: SRI International

(21) Appl. No.: 12/212,646

(22) Filed: Sep. 17, 2008

Related U.S. Application Data
(60) Provisional application No. 60/973,027, filed on Sep. 17, 2007.

(51) Int. Cl. A61K 49/00

(52) U.S. Cl. 424/9.6

(57) ABSTRACT

Tumor boundaries and wound healing are optically imaged by cross-linking a fluorescently-labeled tissue transglutaminase substrate into extracellular matrix.

This application is a continuation of, and claims priority to U.S. Ser. No. 60/973,027, filed Sep. 17, 2007 by the same inventors.

This work was supported by grants from the DOD Breast Cancer Research Program (Grant No. W81XWH-05-1-0386) and the National Cancer Institute (Grant No. 1R21 CA107285); the Government has certain rights in this invention.

### FIELD OF THE INVENTION

The field of the invention is tumor boundary imaging.

### BACKGROUND OF THE INVENTION

Detection and monitoring of tumor boundaries are vital for improving the understanding of cancer progression and for effectively treating cancer. Near-infrared (NIR) fluorescence imaging provides a benign technique for detection by avoiding the use of ionizing radiation or large electromagnetic fields. Specifically anchoring designed NIR fluorescent probe into tumor boundary tissues holds the key for developing this technique.

An enzyme, transglutaminase, TG, catalyzes the formation of ε(γ-glutamyl) lysine bonds (isopeptide bonds) between its substrates. The resulting high molecular weight aggregates are stable and more resistant to proteolytic degradation. While plasma TG, Factor XIII, is active in the fibrin clot formation process, tissue TG is expressed, active and directly involved in rat dermal wound healing and angiogenesis. tTG is also active at the border between normal and malignant tissues helping form neovasculature and stable extracellular matrix. Therefore, mapping TG activity offers a unique mechanism to observe the tumor boundary.

Here we disclose a novel strategy of noninvasive optical imaging by cross-linking a fluorescently-labeled tissue transglutaminase substrate into the extracellular matrix for labeling tumor boundaries and wounds.

### SUMMARY OF THE INVENTION

The invention provides methods and imaging systems and apparatuses for in vivo, non-invasive, whole-animal, fluorescent labeling and detecting of a tissue that is a wound or tumor boundary comprising tissue transglutaminase (tTG), generally comprising the steps of: (a) introducing into the animal a tTG substrate labeled with a near-infrared (NIR) fluorescent tag, under conditions wherein the tTG covalently incorporates the substrate at the tissue; and (b) optically detecting in the animal resultant covalently-incorporated, NIR fluorescent tagged substrate at the tissue. The detecting step is generally deferred from 10 minutes to 0.5, 1, 2, 5 or 10 hrs after the introducing step to allow for label incorporation at the tissue boundary.

The invention encompasses alternative combinations of particular embodiments: the substrate is selected from fibrinogen, NQEQVSP (SEQ ID NO:01), TVQQEL (SEQ ID NO:02), PGGQQIV (SEQ ID NO:03), and GQDPVK (SEQ ID NO:04); the fluorescent tag is a cyanine dye; the tissue is a wound or tumor (solid) boundary; and/or the detecting step is performed using a continuous-wave infrared fluorescence imaging system in a reflection geometry. The method may further comprise repeating the detecting step over time during healing of the wound or change (progress or remission) of the tumor.

### DETAILED DESCRIPTION OF SPECIFIC EMBODIMENTS OF THE INVENTION

Infrared Imaging Systems. The methods are amendable to established noninvasive, whole animal, in vivo optical imaging systems (e.g. Doyle, et al. “In vivo bioluminescence imaging for integrated studies of infection,” Cell Microbiol 6, 303-317 (2004); Ntziachristos, et al. “In vivo tomographic imaging of near-infrared fluorescent probes,” Mol Imaging 1, 82-88 (2002)). In exemplary experiments, we use a continuous-wave infrared fluorescence imaging system in a reflection geometry. Infrared diode lasers are used (instead of the LED array) for illumination. A laser of approximately 1 W is used with wavelength near 795 nm for the carbocyanine dye. Combinations of long-pass and interference filters are used to block the laser beams and transmit the reemitted fluorescence light. We use a CCD camera including a C-mount that allows the lens to be swapped to accommodate different fields of view.

Fluorescent TG Substrates. The methods are amendable to alternative, established TG substrates (e.g. fibrinogen, NQEQVSP (SEQ ID NO:01), TVQQEL (SEQ ID NO:02), PGGQQIV (SEQ ID NO:03), and GQDPVK (SEQ ID NO:04)), and alternative, established NIR fluorescent tags; see, e.g. Bugaj et al., “Novel fluorescent contrast agents for optical imaging of in vivo tumors based on a receptor-targeted dye-peptide conjugate platform,” J. Biomed. Opt. 6, 122-133 (2001); Achilefu et al. “Synthesis, in vitro receptor binding, and in vivo evaluation of fluorescein and carbocyanine peptide-based optical contrast agents,” J. Med. Chem. 45, 2003-2015 (2002); Achilefu et al., “Novel receptor-targeted fluorescent contrast agents for in vivo tumor imaging,” Invest. Radiol. 35, 479-485 (2000); and Chen, et al. “Metabolism-enhanced tumor localization by fluorescence imaging: in vivo animal studies,” Opt. Lett. 28, 2070-2072 (2003). In exemplary experiments, FITC and NIR fluorescence probe, HiLyte Fluor™ 750, were conjugated to TG substrates, fibrinogen and peptide NQEQVSP, via N-hydroxysuccinimide chemistry.

TG Substrate Viability Assays. Viability was tested by: (1) cross-linking into fibrin gel, and (2) reacting with fibrinogen in the presence of Guinea pig liver TG. Our data show that fluorescently-labeled substrate can incorporate into fibrin gel with no inhibition on the initiation of thrombosis. In the presence of Ca2+, cross-linked fibrin clots were formed. The analysis shows that the cross-linking fibrin clot has the strongest intensity, indicating that peptide was crosslinked into the fibrin clot. SDS PAGE shows that, after adding tTG, two of three low MW bands have disappeared and new high MW bands, including the residuals in the wells, have appeared.

Imaging Apparatus. In exemplary animal imaging experiments, the excitation light source was a 10 mw 735 nm laser diode. A digital monochrome CCD camera (Retiga 1300, QImaging) was used to acquire images. A HQ810/90m and a RG665 filter were used to eliminate scattered excitation light. The laser diode light source was shifted laterally to two different positions for image acquisition.

Wound Healing Imaging. Animal imaging was performed on a wound healing model with Fisher rats. After shaving, four 8-mm punch biopsy wounds were created on the upper dorsal region. To examine the timing of injecting fluorescent TG substrate, two injection time points were used. For rat #1, HyLite Fluor™ 750-labeled fibrinogen was injected via tail vein immediately after the punch biopsy and a background image was taken. For rat #2, injection occurred 24 hours after the biopsy.

Clearance Studies. For blood draw studies, rats are injected with labeled substrate (supra) and 200 microliters blood is drawn from the tail vein at 5 minutes after initial injection, and at 1, 4, 8, and 24 hours after injection. The blood is centrifuged and the fluorescence of the supernatant measured using a fluorimeter. For the wound-healing model, animals are injected as described above, punch biopsies are taken at times equal to ½, 1, and 2 times the blood concentration half-life, and infrared imaging is performed at 3-4 hours thereafter. The animals are then sacrificed and the wound region harvested for histological analysis.

Tumor Boundary Imaging. Fibrinogen labeled with rhodamine (3 mg/ml, 200 μl) was given IV to rats with subcutaneous tumors of GFP labeled R3230 mammary adenocarcinoma. Ex vivo images with Zeiss confocal were captured 30 minutes after the injection. The resultant data clearly show that fibrinogen quickly accumulates at the tumor boundary instead of the main tumor mass.

Methods. 1×10⁶ R 3230 mammary carcinoma cells are injected subcutaneously in the thigh region and tumor growth is assessed every 4 days by both caliper measurements and optical imaging. When the tumor reaches a mean volume of 150-200 mm³ substrate is injected into the tail vein. Labeled substrate is administered systemically through the tail vein at three concentrations corresponding to ⅕th, 1 times, and 5 times the concentration used for the wound healing experiments (supra) on each of 3 animals. Imaging is performed on the same day with confirmation by histology. Experiments are performed at concentrations producing 30 to 1 signal-to background levels.

The descriptions of particular embodiments and examples are offered by way of illustration and not by way of limitation. All publications and patent applications cited in this specification and all references cited therein are herein incorporated by reference as if each individual publication or patent application or reference were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

TABLE 1
Summary of animal model experiments

| | | | | | Subsequent | | |
|---|---|---|---|---|---|---|---|
| Animal | | | Procedure | Subsequent | Procedure | Optical | Time of |
| Model | Test # | Control # | at Time 0 | Procedure | Time(s) | Imaging | Harvest |
| Wound | 5 | 3 | Punch | Substrate | Day 1 | Day 1 | Immediate |
| Healing | | | Biopsy | Injection | | | |
| (Punch | | | | | | | |
| Biopsy) | | | | | | | |
| Wound | 5 | 3 | Punch | Substrate | Day 4 | Day 4 | Immediate |
| Healing | | | Biopsy | Injection | | | |
| (Punch | | | | | | | |
| Biopsy) | | | | | | | |
| Wound | 5 | 3 | Punch | Substrate | Day 8 | Day 8 | Immediate |
| Healing | | | Biopsy | Injection | | | |
| (Punch | | | | | | | |
| Biopsy) | | | | | | | |
| Clearance | 19 | 9 | Substrate | Punch | ½, 1 & 2 | 3-4 hrs | Immediate |
| Study | | | Injection | Biopsy | times half | later | |
| (Punch | | | | | life | | |
| Biopsy) | | | | | | | |
| Tumor | 5 | 5 | Tumor | Substrate | Day 0 | Day 0 | Immediate |
| R3230Ac | | | reaches | Injection | | | |
| | | | 200 mm³ | | | | |
| Tumor | 5 | 3 | Tumor | Substrate | Days 0 & 4 | Days 0 | Day 4 |
| R3230Ac | | | reaches | Injection | | & 4 | |
| | | | 200 mm³ | | | | |
| Tumor | 5 | 3 | Tumor | Substrate | Days 0, 4 | Days 0, | Day 8 |
| R3230Ac | | | reaches | Injection | & 8 | 4 & 8 | |
| | | | 200 mm³ | | | | |

What is claimed is:

1. A method for in vivo, whole animal fluorescent labeling and detecting of a tissue that is a wound or tumor boundary comprising tissue transglutaminase (tTG),
(a) introducing into the animal a tTG substrate labeled with a near-infrared (NIR) fluorescent tag, under conditions wherein the tTG covalently incorporates the substrate at the tissue; and
(b) optically detecting in the animal resultant covalently-incorporated, NIR fluorescent tagged substrate at the tissue.

2. The method of claim 1, wherein the substrate is selected from fibrinogen, NQEQVSP (SEQ ID NO:01), TVQQEL (SEQ ID NO:02), PGGQQIV (SEQ ID NO:03), and GQDPVK (SEQ ID NO:04).

3. The method of claim 1, wherein the substrate is fibrinogen.

4. The method of claim 1, wherein the fluorescent tag is a cyanine dye.

5. The method of claim 1, wherein the tissue is a wound.

6. The method of claim 1, wherein the tissue is a wound and the substrate is fibrinogen.

7. The method of claim 1, wherein the tissue is a wound, and the method further comprises repeating the detecting step over time during healing of the wound.

8. The method of claim 1, wherein the tissue is a tumor boundary.

9. The method of claim 1, wherein the tissue is a tumor boundary and the substrate is fibrinogen.

10. The method of claim 1, wherein the detecting step is performed using a continuous-wave infrared fluorescence imaging system in a reflection geometry.

11. The method of claim 1, wherein the substrate is fibrinogen, the fluorescent tag is a cyanine dye.

12. The method of claim 1, wherein the substrate is fibrinogen, the fluorescent tag is a cyanine dye, and the detecting step is performed using a continuous-wave infrared fluorescence imaging system in a reflection geometry.


SEQUENCE LISTING (4 sequences)

SEQ ID NO: 1 - protein, 7 aa, mouse
NQEQVSP

SEQ ID NO: 2 - protein, 6 aa, mouse
TVQQEL

SEQ ID NO: 3 - protein, 7 aa, mouse
PGGQQIV

SEQ ID NO: 4 - protein, 6 aa, mouse
GQDPVK